(19) United States
(12) Patent Application Publication
(10) Pub. No.: US 2003/0044359 A1
(43) Pub. Date: Mar. 6, 2003

(54) Dental care agents provided in the form of single-portion capsules

(76) Inventors: Peter Wuelknitz, Leichlingen (DE); Nicole Duschek, Duesseldorf (DE); Claudia Walther-Strangrecki, Hilden (DE)

Correspondence Address: HENKEL CORPORATION, 2500 RENAISSANCE BLVD, STE 200, GULPH MILLS, PA, 19406, US

(21) Appl. No.: 10/262,154

(22) Filed: Sep. 30, 2002

Related U.S. Application Data
(63) Continuation of application No. PCT/EP01/03163, filed on Mar. 20, 2001.

(30) Foreign Application Priority Data
Mar. 29, 2000 (DE) 100 15 662.2

(51) Int. Cl. A61K 7/16; A61K 7/18

(52) U.S. Cl. 424/49; 424/52

(57) ABSTRACT

Dental care preparations are provided in the form of a portion capsule wherein the capsule material is a water soluble polymer and the capsule contains a flowable preparation containing at least 50% by weight of a humectant, at least one flavoring agent and no more than 10% by weight of water.

### CROSS REFERENCE TO RELATED APPLICATIONS

This application is a continuation under 35 U.S.C. §365(c) and §120 of International Application No. PCT/EP01/03163 filed Mar. 20, 2001 and under § 119 of German Patent Application No. 100 15 662.2 filed Mar. 29, 2000.

### SUMMARY OF THE INVENTION

This invention relates to dental care preparations in the form of a portion capsule for use once only filled with a flowable preparation for the cleaning and care of the teeth or the oral cavity.

Dental care preparations in the form of portion capsules have many advantages. They can be stored in simple storage containers or conveniently removed from special capsule dispensers. Application to a toothbrush is unnecessary; the capsules are directly introduced into the mouth and chewed so that the dental care preparation is released and can then be used to clean the teeth, for example with the aid of a brush. The advantages of such a product for dental care, for example on journeys, are obvious.

### BACKGROUND OF THE INVENTION

Accordingly, toothpastes in portion capsules have been repeatedly proposed in the literature. EP 0 378 956 A1 and DE 4109518 A1 describe dental care preparations of which the capsules consist of gelatine. WO 98/00418 A1 also proposes edible materials, such as gelatine or agar, as capsule materials. Unfortunately, gelatine has considerable disadvantages as a capsule material and is unsuitable for liquid preparations with a high content of humectants such as, for example, glycerol, sorbitol and polyethylene glycol. Capsules such as these lose shape and become soft or even leak in the event of prolonged storage.

Accordingly, the problem addressed by the present invention was to provide a dental care preparation in the form of portion capsules which would have a relatively long shelf life and would be easy to use and of which the capsule would be easy to bite in the mouth, would dissolve to a large extent during teeth cleaning, would be physiologically safe and would have a neutral taste.

It has been found that water-soluble polymers from the group of cellulose ethers, polyvinyl alcohol, polyethylene oxide and blends of these polymers are more suitable as a capsule material for holding liquid tooth cleaning preparations containing humectants of the polyol type.

### DESCRIPTION OF THE INVENTION

The present invention relates to dental care preparations in the form of a portion capsule of a water-soluble material filled with a flowable preparation for the cleaning and/or care of the teeth and/or the oral cavity, characterized in that the capsule material consists completely or predominantly of a water-soluble polymer selected from cellulose ethers, polyvinyl alcohol, polyethylene oxide and mixtures thereof and in that the flowable preparation contains at least 50% by weight of a humectant from the group of glycols, glycol ethers and polyols containing 2 to 6 carbon atoms, polyalkylene glycols and mixtures thereof, at least one flavoring agent and no more than 10% by weight of water.

Capsule materials suitable for the purposes of the invention are known from the literature. WO 99/40156 A1, for example, describes an alkylene oxide polymer composition which is suitable for the production of films and capsules. A capsule material based on hydroxypropyl methyl cellulose is known from EP 714 656 A1. EP 592 130 A2 describes a material for hard capsules based on hydroxypropyl methyl cellulose. Finally, a capsule material based on a polymer blend of cellulose ethers and polyvinyl alcohol is known from EP 180 287 A2.

According to the invention, the capsule material consists completely or predominantly of cellulose ethers, polyvinyl alcohol and/or polyethylene oxides. “Predominantly” means that less than 10% by weight of the capsule material should consist of auxiliaries intended to soften or harden the capsule material. The water-soluble polymers suitable for the purposes of the invention may be processed either to soft capsules or to hard capsules. Hard capsules are produced by known methods, for example by the immersion methods described in EP 714 656 A1 and EP 592 130 A2. For the production of soft or flexible capsules, the water-soluble polymer materials are first processed to films. The films are then processed to capsules by the same methods as used for the production of soft gelatine capsules (cf. W. Fahrig, U. Hofer: Die Kapsel, WVT mbH Stuttgart 1983). In these processes, the films are shaped, for example, into pocket-like structures which are then filled with the particular product and sealed with a second film. Examples of such processes are, for example, the Colton process and the Upjohn process. In the Norton encapsulation process, the capsules are shaped between two dies. In the upper part of the mold, the capsule is preformed into a tube from two films and filled through a small filling tube, after which the filled tube is closed by a stamping operation in the lower part to form the capsule.

The Accogel process uses a rotating shaping roller and a vacuum to draw the film into the mold. The particular product to be encapsulated is introduced into the pocket. By pressing on a second film by a second shaping roller, the capsules are closed and stamped out. The so-called rotary die process developed in Detroit (USA) in 1933 by R. P. Scherer works in very much the same way. This process uses two contra-rotating shaping rollers and a filling wedge. Initially, pocket-like structures are formed by welding of the lower and lateral seams and are then filled with the particular product by means of dosing pumps and fine filling channels. As the shaping rollers rotate, the capsule is also welded on top and ejected downwards.

A process adapted for other capsule materials is described in WO 97/35537 A1 (Bioprogress Technology Ltd.). In this process, the films are wetted with a solvating solvent to improve the welding or bonding of the film material to form a leakproof capsule seam.

In a preferred embodiment of the invention, the capsule material consists completely or predominantly of methyl hydroxypropyl cellulose and is processed, preferably in the form of a 0.2 to 1 mm thick and more particularly 0.08 to 0.2 mm thick film, to form capsules with a commensurate wall thickness and a holding capacity of 0.5 to 2 ml.

The described processes for producing capsules from films also enable two films differing in color or transparency to be used for capsule production. This can be achieved, for example, by small quantities of dyes or pigments in the capsule material. In a preferred embodiment of the invention, therefore, the capsule material consists of two half shells differing in color or transparency.

The technology similar to the rotary die process may also be further developed by insertion of a third film as a partition between the two films forming the capsule walls so that portion capsules with two separate compartments are obtained. These compartments can be filled with flowable preparations differing in composition from one another. Not only may these preparations differ in color and transparency, one of the two compartments, for example, may be filled with a liquid preparation and the other with a flowable, powder-form or particulate preparation. For example, two incompatible components may be introduced into a single portion capsule by incorporating them in two preparations of different composition which are introduced into the separate compartments of the two-compartment portion capsule.

In a preferred embodiment, the portion capsule is divided in two by a partition of the same material and at least one of the two compartments is filled with a flowable preparation containing at least 50% by weight of a humectant. In this embodiment, the second compartment is preferably filled with a powder-form, free-flowing composition. This composition may contain, for example, a water-binding component, for example silica gels, so that the stability of the capsules to atmospheric moisture is improved. The second compartment of the two-compartment portion capsule may also be filled, for example, with a powder-form preparation containing active principles sensitive to hydrolysis or oxidation such as, for example, ascorbic acid, coenzyme Q10 or active principles from plants such as chamazulene for example. The powder-form preparation may also be formulated as an effervescent powder, i.e. with a content of a carbonate or bicarbonate salt and a powder-form acid, for example citric acid, or a water-soluble hydrogen citrate salt. On contact with water or saliva, a pleasant prickling and refreshing sensation is experienced in the mouth.

The powder-form compositions preferably contain typical toothpaste abrasives such as, for example, calcium carbonate, dicalcium phosphates, silicas, aluminium hydroxide and/or aluminium oxide, zirconium silicate, pumice stone powder or other inert particulate materials which, by virtue of their texture, are suitable for use for cleaning the teeth. Examples of such components are cellulose powder, kieselguhr, talcum, layer silicates, powdered plastics, pigments or ground parts of plants.

In a preferred embodiment, active principles which are readily absorbed and inactivated by other toothpaste ingredients, for example cationic antibacterial agents, such as chlorhexidine, hexetidine and cetyl pyridinium chloride, may also be separately formulated in the second compartment.

Finally, ingredients which react with particulate constituents of the adjacent compartment to form finely crystalline deposits, such as soluble calcium salts for example, which then react during teeth cleaning with soluble fluorides or phosphates to form fine-particle calcium phosphates or calcium fluorophosphates, such as apatite, hydroxylapatite or fluoroapatite, may be accommodated in the second compartment.

The dental care preparation according to the invention contains a substantially water-free or low-water preparation containing at least 50% by weight of a humectant from the group of water-soluble glycols, glycol ethers or C₂₋₆ polyols, polyalkylene glycols or a mixture thereof as the flowable preparation for the cleaning and/or care of the oral cavity and/or the teeth.

Suitable water-soluble glycols are ethylene glycol, propanediols and butanediols. Suitable glycol ethers are, for example, ethyl glycol, ethyl diglycol, diethylene glycol, triethylene glycol and dipropylene glycol. Suitable polyols are, for example, glycerol, erythritol, diglycerol, xylitol, arabitol, sorbitol, mannitol, dulcitol. Suitable polyalkylene glycols are, above all, the polyethylene glycols and polypropylene glycols and products of the addition of ethylene oxide onto propylene glycol or onto polypropylene glycols. Polyalkylene glycols with an average molecular weight of no more than 1000D are particularly suitable.

At least one flavoring agent is present as another compulsory component of the flowable preparation. Suitable flavoring components are, for example, sweeteners and/or flavoring oils. Suitable flavoring oils are any of the natural and synthetic flavors typically used in oral and dental care preparations. Natural flavors may be used both in the form of the essential oils isolated from the drugs and in the form of the individual components isolated therefrom. The preparation should preferably contain at least one flavoring oil from the group consisting of peppermint oil, spearmint oil, anise oil, Japanese anise oil, caraway oil, eucalyptus oil, fennel oil, cinnamon oil, clove oil, geranium oil, sage oil, pimento oil, thyme oil, marjoram oil, basil oil, citrus oil, gaultheria oil or one or more components of these oils isolated from them or synthetically produced. The most important components of the oils mentioned are, for example, menthol, carvone, anethol, cineol, eugenol, cinnamaldehyde, caryophyllene, geraniol, citronellol, linalool, salvia, thymol, terpinene, terpineol, methyl chavicol and methyl salicylate. Other suitable flavors are, for example, menthyl acetate, vanillin, ionone, linalyl acetate, rhodinol and piperitone.

Suitable sweeteners are, for example, saccharin sodium, acesulfam, aspartame, sodium cyclamate, steviosides, thaumatine, sucrose, lactose, maltose, fructose and glycyrrhizin. The flavoring components may be present in the preparation in quantities of 0.01 to 2% by weight. The flowable dental care preparation is preferably water-free. However, relatively small amounts of water in the formulation are not detrimental to the stability of the capsule membrane. The water content should not exceed 10% by weight.

In addition to the compulsory components mentioned above, the flowable preparation may contain other components of use for cleaning the teeth and gums and keeping them healthy. In a preferred embodiment, these additional components are polishing agents, fluorine compounds and antimicrobial agents. Polishing agents support the mechanical cleaning of the tooth surface during brushing of the teeth.

Basically, suitable polishing agents are any of the known toothpaste abrasives such as, for example, chalk, calcium pyrophosphate, dicalcium phosphate dihydrate, silicas, aluminium hydroxide, aluminium oxide, sodium aluminium silicates, organic polymers and mixtures of these abrasives. More strongly abrasive polishing agents, such as pumice stone powder or zirconium silicate, may also be used in small quantities of no more than 1%. The total content of polishing components is preferably in the range from 5 to 30% by weight, based on the flowable preparation.

Polishing agents of the silica type are particularly suitable for the dental care preparations according to the present invention. Suitable silicas are, for example, silica gels, silica hydrogels and precipitated silicas. Silica gels are obtained by reacting sodium silicate solutions with strong aqueous mineral acids to form a hydrosol, ageing to form the hydrogel, washing and drying. If drying is carried out under moderate conditions to a water content of 15 to 35% by weight, the so-called silica hydrogels known, for example, from U.S. Pat. No. 4,153,680 are obtained. Drying to water contents below 15% by weight results in irreversible shrinkage of the previously loose structure of the hydrogel to the dense structure of the so-called xerogel. Silica xerogels are described, for example, in U.S. Pat. No. 3,538,230.

A second particularly suitable group of silica polishing agents are the precipitated silicas. Precipitated silicas are obtained by precipitation of silica from dilute alkali metal silicate solutions by addition of strong acids under conditions which preclude aggregation to the sol and gel. Suitable processes for the production of precipitated silicas are described, for example, in DE-OS 25 22 486 and in DE-OS 31 14 493. A particularly suitable precipitated silica is that produced in accordance with DE-OS 31 14 493 which has a BET surface of 15 to 110 m²/g, a particle size of 0.5 to 20 μm (at least 80% by weight of the primary particles should be below 5 μm in size) and a viscosity in the form of a 30% glycerin/water (1:1) dispersion of 30 to 60 Pa.s (20° C.) and which is used in a quantity of 10 to 20% by weight, based on the toothpaste. In addition, particularly suitable precipitated silicas of this type have rounded corners and edges and are commercially obtainable under the name of Sident®12 DS (DEGUSSA).

Other precipitated silicas of this type are Sident 8 (DEGUSSA) and Sorbosil AC 39 (Crosfield Chemicals). These silicas are distinguished by a weaker thickening effect and a slightly larger mean particle size of 8 to 14 μm for a specific BET surface of 40 to 75 m²/g and are particularly suitable for liquid preparations.

By contrast, preparations which have a higher viscosity require a sufficiently high percentage content of silicas with a particle size of less than 5 μm, preferably at least 3% by weight of a silica with a particle size of 1 to 3 μm. Accordingly, besides the precipitated silicas mentioned, even finer so-called thickening silicas with a BET surface of 150 to 250 m²/g, for example the commercial products Sipernat 22 LS or Sipernat 320 DS, are preferably added to such preparations.

Another polishing component which may be present in a quantity of about 1 to 5% by weight is, for example, aluminium oxide in the form of lightly calcined alumina containing γ-and α-aluminium oxide. A suitable aluminium oxide such as this is commercially obtainable under the name of “Poliertonerde P10 feinst” (Giulini Chemie).

Fluorine compounds are used to harden the enamel and hence to prevent caries. The dental care preparations according to the invention may contain sodium fluoride, zinc fluoride, tin(II) fluoride, amine fluoride or sodium monofluorophosphate, for example, as the fluorine compounds. A quantity of 0.01 to 0.2% by weight fluorine in the form of the compounds mentioned should preferably be present.

Antimicrobial compounds are effective against the protein-and starch-degrading and acid-forming bacteria of dental plaque and against the particularly obstinate germs of chronic gingivitis. Accordingly, they prevent the formation of halitosis, caries and parodontitis. Suitable antimicrobial compounds are, for example, cationic surfactants such as, for example, cetyl trimethyl ammonium bromide, benzethonium chloride, cetyl pyridinium chloride or the N,N,N′-tris-(2-hydroxyethyl)-N′-octadecyl-1,3-diaminopropane dihydrofluoride known as amine fluoride. Also suitable are the antimicrobial biguanide compounds such as, for example, polyhexamethylene biguanide (Vantocil® IB, ICJ) or 1,1′-hexamethylene-bis-(4-chlorophenyl)-biguanide (“chlorhexidine”) in the form of a water-soluble compatible salt, for example in the form of the acetate or gluconate. The antimicrobial 5-aminohexahydropyrimidines, for example 1,3-bis-(2-ethylhexyl)-5-methyl-5-aminohexahydropyrimidine (“hexetidine”), are also particularly suitable, as are non-cationic, phenolic antimicrobial compounds, more particularly halogenated phenols and diphenylethers. Particularly suitable antimicrobial compounds of this type are, for example, 6,6′-methylene-bis-(2-bromo-4-chlorophenol) (“bromochlorophene”) and 2,4,4′-trichloro-2′-hydroxydiphenylether (“triclosan”).

Other suitable antimicrobial agents are the p-hydroxybenzoic acid esters and sesquiterpene alcohols such as, for example, bisabolol, farnesol, santalol and nerolidol. Antimicrobial agents may be present in the dental care preparations according to the invention in a quantity of 0.005 to 0.5% by weight, based on the flowable preparation.

Besides these preferred components, the dental care preparations according to the invention may also contain other active principles and auxiliaries commonly used in dental care preparations such as, for example,

wound-healing and anti-inflammatory agents such as, for example, allantoin, urea, azulene, camomile-based active principles and acetylsalicylic acid derivatives;

vitamins such as, for example, retinol or retinol esters, ascorbic acid derivatives, panthenol, biotin,

desensitizing components such as, for example, potassium or strontium salts, eugenol, clove oil,

remineralizing salts such as, for example, calcium and magnesium salts, more particularly phosphates,

anti-scale agents such as, for example, condensed phosphates, for example sodium pyrophosphate, sodium tripolyphosphate, organophosphonates such as, for example, the sodium salt of 1-hydroxyethane-1,1-diphosphonic acid, 1-phosphonopropane-1,2,3-triphosphonic acid or azacycloheptane-2,2-diphosphonic acid, phosvitin, tranexamic acid and other complexing agents.

Suitable auxiliaries for adjusting consistency or pH, color and transparency are, for example,

binders such as, for example, cellulose, cellulose ethers, starch and starch ethers, biopolymers such as, for example, xanthan gum, vegetable gums such as, for example, agar agar, carrageen, tragacanth, guar, acacia gum, locust bean gum, pectins and synthetic polymers such as, for example, polyvinyl pyrrolidone, polyvinyl alcohol and carboxyvinyl polymers,

inorganic thickeners such as, for example, colloidal silica (Aerogel silica, pyrogenic silicas), layer silicates (clays, montmorillonite),

dyes, pigments, for example titanium dioxide,

buffering agents such as, for example, citric acid/sodium citrate or mixtures of primary, secondary or tertiary alkali metal phosphates.

In addition, surfactants and lower alcohols may be present in relatively small quantities of, in all, no more than 3% by weight in order to improve cleaning performance and for stably emulsifying or solubilizing the flavoring components.

The addition of a surfactant may also be desirable for producing a foam during brushing of the teeth, for stabilizing the dispersion of polishing agents and for emulsifying or solubilizing the flavoring oils. Suitable surfactants which develop a certain foaming effect are the anionic surfactants, for example sodium alkyl sulfates containing 12 to 18 carbon atoms in the alkyl group. These surfactants also have a certain enzyme-inhibiting effect on the bacterial metabolism of plaque. Other suitable surfactants are alkali metal salts, preferably sodium salts, of alkyl polyglycol ether sulfate containing 12 to 16 carbon atoms in the linear alkyl group and 2 to 6 glycol ether groups in the molecule, of linear alkane (C₁₂₋₁₈) sulfonate, of sulfosuccinic acid monoalkyl (C₁₂₋₁₈) esters, of sulfated fatty acid monoglycerides, sulfated fatty acid alkanolamides, sulfoacetic acid alkyl (C₁₂₋₁₆) esters, acyl sarcosines, acyl taurides and acyl isethionates containing 8 to 18 carbon atoms in the acyl group.

Zwitterionic and ampholytic surfactants may also be used, preferably in combination with anionic surfactants. However, it is particularly preferred to use nonionic surfactants to promote the cleaning effect. Suitable nonionic surfactants are, for example, products of the addition of ethylene oxide onto fatty alcohols, onto fatty acids, onto fatty acid monoglycerides, onto sorbitan fatty acid monoesters or onto methyl glucoside fatty acid monoesters. The quantity of ethylene oxide added on should be so large that the surfactants are soluble in water, i.e. at least 1 g/l should be soluble in water at 20° C. Another group of suitable surfactants are the alkyl (oligo)-glycosides containing 8 to 16 carbon atoms in the alkyl group and having a degree of oligomerization of the glycoside unit of 1 to 4. Alkyl (oligo)glyco-sides, their production and use as surfactants are known, for example, from U.S. Pat. No. 3,839,318, DE-A-20 36 472, EP-A-77 167 or WO-A-93/10132.

So far as the glycoside unit is concerned, monoglycosides (x=1) where a monosaccharide unit is attached to a C₁₀₋₁₆ fatty alcohol by a glycoside linkage and oligomeric glycosides with a degree of oligomerization x of up to 10 are suitable. The degree of oligomerization is a statistical mean value on which the homolog distribution typical of such technical products is based.

A particularly suitable alkyl (oligo)glycoside is an alkyl (oligo)glucoside with the formula RO(C₆H₁₀O)ₓ—H, where R is an alkyl group containing 12 to 14 carbon atoms and x has a mean value of 1 to 4.

A nonionic solubilizer from the group of surface-active compounds may be necessary, particularly for solubilizing the generally water-insoluble flavoring oils. Particularly suitable nonionic solubilizers are, for example, ethoxylated fatty acid glycerides, ethoxylated fatty acid sorbitan partial esters or fatty acid partial esters of glycerol or sorbitan ethoxylates. Solubilizers from the group of ethoxylated fatty acid glycerides include above all products of the addition of 20 to 60 moles of ethylene oxide onto mono-and diglycerides of linear fatty acids containing 12 to 18 carbon atoms or onto triglycerides of hydroxy fatty acids, such as hydroxystearic acid or ricinoleic acid. Other suitable solubilizers are ethoxylated fatty acid sorbitan partial esters, i.e. preferably products of the addition of 20 to 60 moles ethylene oxide onto sorbitan monoesters and sorbitan diesters of fatty acids containing 12 to 18 carbon atoms. Other suitable solubilizers are fatty acid partial esters of glycerol or sorbitan ethoxylates, i.e. preferably monoesters and diesters of C₁₂₋₁₈ fatty acids and products of the addition of 20 to 60 moles ethylene oxide onto 1 mole glycerol or onto 1 mole sorbitol. Ethanol or isopropanol in a quantity of 0.1 to 2% by weight may be present as lower alcohols.

The following Examples are intended to illustrate the invention:

### EXAMPLES

1. Tooth cream formulations for MHPC capsules

| | 1.1 | 1.2 |
|---|---|---|
| Glycerol (99.5%) | 40.0 | 93.7 |
| Sorbitol (100%) | 15.0 | — |
| 1,2-Propylene glycol | 17.0 | — |
| Polyethylene glycol 400 | 2.0 | 3.0 |
| Keltrol F | 0.2 | 0.9 |
| Sident 8 | 12.0 | — |
| Sident 22 S | 4.5 | — |
| Silica FK 320 DS | 1.0 | — |
| Saccharin Na | 0.25 | — |
| Flavoring oil | 0.1 | 0.1 |
| Tagat S | 0.1 | 0.1 |
| Na dihydrogen phosphate | — | 0.2 |
| Na dihydrogen citrate | — | 0.2 |
| Dye (green), CI 74260 | — | 0.01 |
| Water | to 100 | to 100 |

1 ml portion capsules of methyl hydroxypropyl cellulose with a wall thickness of 0.1 mm were filled with the compositions by the process described in WO 97/35537 A1. The capsules obtained were unchanged after dry storage for 6 weeks at 25° C.

The following commercial products were used:

| | |
|---|---|
| Keltrol F (Kelco): | xanthan gum |
| Sident 8 (Degussa): | synth. amorphous silica, BET: 60 m²/g |
| Sident 22 S (Degussa): | synth. amorphous silica, BET: 140 m²/g |
| Silica FK 320 DS (Degussa): | synth. amorphous silica, BET: 170 m²/g |
| Tagat S (Tego Cosmet.): | PEG 30 Glyceryl Stearate |

2. Powder formulation for a two-compartment portion capsule

The first compartment contains the tooth cream of Example 1.1 or 1.2. The second compartment contains a powder composition to formulation 2.1, 2.2 or 2.3.

| | 2.1 | 2.2 | 2.3 |
|---|---|---|---|
| Calcium carbonate | 95.0 | — | — |
| Dicalcium phosphate dihydrate | — | — | 98.2 |
| Cellulose powder | — | 99.0 | — |
| Sodium hydrogen carbonate | 5.0 | — | — |
| Sodium hydrogen phosphate | — | — | 1.0 |
| Sodium dihydrogen phosphate | — | — | 0.5 |
| Sodium fluoride | — | — | 0.3 |
| Camomile, powdered | — | 1.0 | — |

What is claimed

1. A dental care preparation in the form of a portion capsule of a water-soluble material filled with a flowable preparation for the cleaning and/or care of the teeth and/or the oral cavity, wherein the capsule material comprises, completely or predominantly, a water-soluble polymer selected from the group consisting of cellulose ethers, polyvinyl alcohol, polyethylene oxide and mixtures thereof and further wherein the flowable preparation contains at least 50% by weight of a humectant selected from the group consisting of glycols, glycol ethers or polyols containing 2 to 6 carbon atoms, polyalkylene glycols or a mixture thereof, at least one flavoring agent and no more than 10% by weight of water.

2. The dental care preparation of claim 1, wherein the capsule material consists completely or predominantly of a methyl hydroxypropyl cellulose.

3. The dental care preparation of claim 2, wherein the methyl hydroxypropyl cellulose is in the form of a 0.2 to 1 mm thick film.

4. The dental care preparation of claim 2, wherein the capsule material consists of two half shells differing in transparency or color.

5. The dental care preparation of claim 2, wherein the portion capsule is divided in two by a partition of the same material and at least one of the two compartments is filled with the flowable preparation of claim 1.

6. The dental care preparation of claim 5, wherein the second compartment is filled with a flowable powder composition.

7. The dental care preparation of claim 6, wherein the flowable powder composition additionally contains a water-binding component.

8. The dental care preparation of claim 2, wherein the flowable preparation additionally contains polishing agents, fluorine compounds and/or antimicrobial agents.

9. The dental care preparation of claim 3 wherein the capsule has a holding capacity of from 0.5 to 2 ml.
